(19) United States
(12) Patent Application Publication
(10) Pub. No.: US 2014/0276337 A1
(43) Pub. Date: Sep. 18, 2014

(54) Implantable Devices with Delivery Systems and Methods for Blocking Digestive Neurohormonal Pathways in Mammals

(71) Applicant: MetaTech Life Sciences, LLC, Redwood City, CA (US)

(72) Inventors: Michael Lawrence Reo, Redwood City, CA (US); Darin Howard Buxbaum, Palo Alto, CA (US)

(21) Appl. No.: 14/214,769

(22) Filed: Mar. 15, 2014

Related U.S. Application Data
(60) Provisional application No. 61/791,194, filed on Mar. 15, 2013.

(51) Int. Cl. A61F 5/00

(52) U.S. Cl. CPC A61F 5/0076
USPC 604/8

(57) ABSTRACT

Described herein are implantable devices, delivery systems and surgical methods for creating a barrier that may be deployed into a digestive system organ to block the biochemical and/or neurohormonal systems thereby providing therapeutic benefit ideally treating diabetes mellitus and obesity. The device for creating a barrier in the digestive system organ may include a wall or conduit whether impermeable or porous at varying degrees. Method of surgical delivery for the device described herein may be conducted through open, laparoscopic, minimally invasive, or endoscopic means. The device may be temporarily secured on a novel delivery system enabling the methods described. The device may be secured to the digestive organ allowing natural biomechanical motion (peristaltic motion) of the digestive system without untoward effects.

### CROSS-REFERENCE TO RELATED APPLICATIONS

This application claims the benefit of U.S. Provisional Patent Application Ser. No. 61/791,194 entitled “Implantable Devices with Delivery Systems and Methods for Blocking Digestive Neurohormonal Pathways in Mammals,” filed Mar. 15, 2013, from which priority is claimed under 35 U.S.C. 119, and the disclosure of which is hereby incorporated herein by reference in its entirety.

### TECHNICAL FIELD

The inventions described herein include devices, systems, and methods for blocking various neurohormonal pathways of the digestive system, ideally treating diabetes mellitus and/or obesity. More specifically, described herein are devices, delivery systems, and surgical methods for deploying implantable devices in a mammalian or human gastrointestinal tract that may mechanically and/or chemically block the digestive system's neurohormonal and/or biochemical pathways that can elicit undesirable physiological responses in the mammal/human, which can be used to address a variety of conditions, including for the treatment of diabetes mellitus and/or obesity.

### BACKGROUND OF THE INVENTION

According to the World Health Organization, 347 million people worldwide suffer from diabetes mellitus Type I and Type 2. Diabetes has detrimental characteristics such as insulin resistance, inadequate insulin secretion, amyloid formation in islet tissue, and decreased number of beta cells. As such, diabetes is predicted to be in the top ten leading causes of death. In addition, mammals such as felines suffer from diabetes mellitus exhibiting similar characteristics as humans (Henson et al., Feline Models of Type 2 Diabetes Mellitus, ILAR Journal, 2006). According to literature, about one in 400 canines and about one in every 200 feline's suffer from diabetes.

Regarding obesity, over 20% of the US population is obese according to the Center of Disease Control (CDC). Obesity increases the risk of a number of health conditions including heart disease, stroke, Type 2 Diabetes (T2D), adverse lipid concentrations and some forms of cancer. As a result, the U.S. spends an estimated annual cost of $147 billion for treating obesity, and on average the medical costs for people who are obese were higher than those of normal weight. Unfortunately, obesity may not be confined to just humans. New studies have revealed increased rates of obesity in mammals, ranging from feral rats to domestic pets and laboratory primates. Approximately 50% of adult cats and dogs were classified as overweight or obese by their veterinarians. Also, a similar list of health dangers as compared to humans comes with the excess weight, including the shortening of a pet's life.

Since the prevalence of obesity and diabetes are high in the U.S., the U.S. has spent significant efforts in researching and identifying the physiological causes of T2D and obesity in an attempt to provide various treatment options. Research has shown that T2D and obesity may be caused by physiological actions from the small bowel. The primary function of the small bowel is the chemical digestion of food and the absorption of proteins, lipids (fats) and carbohydrates (i.e., the nutrients). The small bowel senses the presence of the nutrients and sends a signal to the brain. The brain responds by releasing hormones that trigger the endocrine system to release other hormones in the small bowel that may stimulate or inhibit insulin and glucagon production while the small bowel completes the enzymatic breakdown of the ingested food to isolate the nutrients (proteins, fats and carbohydrates). The nutrients are absorbed through the wall of the small bowel through diffusion into the blood stream, where the body regulates the disposition of the nutrients. The body then activates a counter-regulatory system by releasing another set of hormones that can act to balance the sugar levels in the bloodstream. This is thought to be the mechanism that regulates blood glucose levels, eventually influencing diabetes and/or obesity. The food that remains undigested and unabsorbed passes into the large intestine. (Sarruf et al., New Clues to Bariatric Surgery's Benefits, Nature Medicine, Volume 18, Number 6; June 2012) Similar regulatory and counter-regulatory systems are shared in most mammals.

Armed with this knowledge, there have been many attempts at therapies and invasive procedures created to treat T2D and obesity in humans and mammals. Such medical therapies and invasive treatment options have included the use of pharmaceuticals, insulin replacement, diets, exercise, gastric bypass, vertical banded gastroplasty, Roux-en-Y, and/or adjustable gastric banding. These therapies and invasive treatments have documented results, but have potentially serious physical and physiological complications because they may interfere with the digestive regulatory and counter regulatory systems (i.e., digestive biomechanics) by removing a portion of the small intestine or mechanically affecting the small intestine. Complications may arise from this interference, such as “dumping syndrome,” dehiscence (separation of tissue that was stapled together), leaks from staple lines, changes in quality of life, stretching of the stomach, revision surgery, diarrhea, vomiting, infection, sepsis and/or various other complications.

### BRIEF SUMMARY OF THE INVENTION

Although, many therapies and invasive treatments exist for diabetes and obesity, none have resolved these various diseases without significant complications or life style changes. There remains an unaddressed need for an invention that remedies these disadvantages for diabetic and/or obese humans and mammals. The invention disclosed herein describes implantable devices, delivery systems, and methods for blocking some or all of the digestive system's relevant neurohormonal pathways to treat diabetes and obesity, while not squeezing, cinching, removing and/or resecting the small bowel and/or interfering with small bowel digestive biomechanics in an untoward manner.

In one embodiment, the gastrointestinal (GI) system may comprise an implant, a delivery system, and a novel surgical technique.

In some embodiments, the implant may be made from a biomaterial that mechanically blocks various digestive tissue(s) from contacting bolus of matter with nutritional value. In some embodiments, the blocking implant may be made from expanded polytetrafluoroethylene (ePTFE) and polyethylene terephthalate (PET) reinforced-silicone (PDMS). In some embodiments, the implant may include a tissue in-growth promoting material and/or chemical coatings to block neurohormonal pathways while remaining minimal in physical mass and/or surface area for the benefit of biologic integration and toleration.

In some embodiments, the implant may be made from a deformable or suitably flexible material. In some embodiments, the implant may be made from silicone, polyethylene terephthalate reinforced silicones, fluoropolymers, polycarbonate urethanes with or without functional end groups, aromatic or aliphatic polyurethanes, or polymeric fabrics such as meshes woven or knit, and/or any combination thereof. In some embodiments, features of the implant may be metallic or an alloy such as marker bands for radiologic imaging or for securement purposes. In some embodiments, the implant may incorporate a tissue ingrowth media. In some embodiments, the implant porosity or thin wall transmission may include a variety of features for eluting therapeutic chemistry.

In some embodiments, the wall of the implant may be overall thin, locally thin or appropriately porous in various locations to facilitate discharge of therapeutic chemistry and/or exchange of nutrients to the distal digestive system by varying degrees or amount of passage, thereby eliciting degrees of effect on the biochemistry. Specifically, embodiments described herein may induce effects that directly affect the blood glucose and/or insulin chemistry, or such embodiments may induce effects that indirectly influence such physiological conditions by interference and/or other action relative to the neurohormonal pathways. In some embodiments, the wall or body of the implant is composed of and/or comprises at least two layers, such that one layer provides mechanical reinforcement, such as previously described, and another layer provides chemistry critical to the therapeutic treatment of diabetes or obesity. Alternatively, a device for creating a barrier into the digestive system organ may include a wall, wherein the thickness and/or porosity of the wall can be defined to a size and/or degree such that it creates a barrier between the nutrients and the digestive system organ and/or portions thereof.

In some embodiments, the implant can include universal and/or adjustable features that allow adjustment of the implant to accommodate the size of the organ and/or organ portion in question. In some embodiments, the implant can be secured in one location to the digestive system. In some embodiments, the implant can be secured to the digestive system in at least two locations, such as at a proximal location and a distal location of the implant, or at multiple locations such as corners of a polygon or circular wall. In some embodiments, the implant may include features that allow passage of digestive constituents while maintaining distal securement to at least one digestive organ. In some embodiments, the implant is retained by a fiber, sutures and/or pledget material comprising appropriate biomaterials promoting ingrowth. In some embodiments, the device may cylindrically interface with tissue, and may be fixedly attached yet be reversible and/or removable in at least one location.

In some embodiments, the implant can be delivered using a replicated holding-feature delivery system, including systems that incorporate no moving components, which can include a solid-state system specifically (i.e., a fixed, one-piece unit). In some embodiments, the implant could be delivered by a single holding-feature. The holding-feature may contain one or more novel features that allow tactile interpretation and location of the correct position to secure the implant. In some embodiments, the delivery system may temporarily hold the implant by suture, elastic, or viscoelastic retainer or connector. The retainer or connector may or may not be integrated with the implant and/or holding-feature.

In some embodiments, the delivery system is manufactured or assembled modularly. Specifically, each component can be exchanged for a different size component, as necessary. If desired, one or more components could be assembled (but not necessarily limited to such assembly) in a controlled environment such as white room, laboratory, controlled environment room, cleanroom, clinical environment, or operating room.

In some embodiments, the delivery system can be fabricated as a single unit or multiple components assembled in a single step such as injection molding. In various embodiments, the device and/or delivery system could be packaged sterile or unsterile as a kit.

In some embodiments, the delivery system may comprise at least one securing feature. The securing feature may comprise a length of material intended to extend between the implant and another securing feature. If desired, a securing feature could include a holding feature, a retaining feature, a connecting feature, or a fixation feature, each of which could include the intention of fixedly attaching one feature to another feature, ideally securing implant components to the tissue or biological constituent. In some embodiments, at least one securing feature is integrated with the implant features or retaining material.

In some embodiments, the device could be implanted through a plurality of surgical techniques. Such surgical techniques could include open surgical, laparoscopic, endoscopic, and/or minimally invasive procedures, or various combinations thereof.

### BRIEF DESCRIPTION OF THE SEVERAL VIEWS OF THE DRAWINGS

FIGS. 1A-1C depict various exemplary views of one embodiment of an implantable device constructed in accordance with various teaching of the present invention;

FIGS. 2A-2D depict various exemplary views of an alternate embodiment of an implantable device;

FIGS. 3A-3B depicts an isometric view of various embodiments of cuffs for an implantable device;

FIGS. 4A-4B depict various exemplary views of another alternate embodiment of an implantable device with retaining mechanism cuffs;

FIGS. 4C-4D depict magnified isometric views of the retaining mechanism cuffs of the implantable device of FIGS. 4A-4B;

FIGS. 5A-5B depicts various exemplary views of an implantable device cuff retaining ring;

FIGS. 6A-6B depicts various exemplary views of an implantable device cuff pledget;

FIG. 7 depicts an isometric view of one embodiment of a GI delivery system;

FIGS. 8A-8C depicts various exemplary views of the GI delivery system handle of FIG. 7;

FIGS. 9A-9B depicts various exemplary views of embodiments of attachment rods for use with a GI delivery system;

FIGS. 10A-10C depicts various exemplary views of one embodiment of a large sized GI delivery system holding component nose cone;

FIGS. 11A-11C depicts various exemplary views of one embodiment of a medium sized GI delivery system holding component nose cone;

FIGS. 12A-12C depicts various exemplary views of one embodiment of a small sized GI delivery system holding component nose cone;

FIGS. 13A-13B depict various exemplary views of the GI delivery system of FIG. 7 with an associated implantable device;

FIG. 14 illustrates an exemplary mammalian upper digestive tract; and

FIG. 15 illustrates one embodiment of an implantable device positioned within the small bowel.

### DETAILED DESCRIPTION OF THE INVENTION

Components

Described herein are variations of implantable devices, systems, and methods for creating a chronically stable biomechanically suitable neurohormonal barrier into a digestive system organ. More specifically, described herein is a GI delivery system that may comprise an implantable device and/or liner 10, a delivery system and/or a novel surgical technique.

FIGS. 1A-1C depict various exemplary views of one embodiment of an implantable device and/or liner 10. The implantable liner 10 may include a proximal cuff 20, a distal cuff 25 and a conduit 30 as shown in FIG. 1A. The proximal cuff 20 and distal cuff 25 may comprise a cuff inner diameter 100 and a cuff outer diameter 90, where the cuff inner diameter 100 may be larger than the conduit inner diameter 110 (see FIG. 1C). Such an increased cuff inner diameter 100 may allow large boluses of food to enter the cuff inner diameter 100 and facilitate flow digested food through the smaller conduit inner diameter 110. In alternative embodiments, the conduit inner diameter may be equal to and or larger than the cuff inner diameter, if desired. In a similar manner, the proximal and/or distal cuffs may be of equal or differing sizes.

Alternatively, in other embodiments, the cuff outer diameter 90 may be designed in a variety of shapes and/or sizes, including at least one embodiment in which the cuff outer diameter 90 is formed larger than the corresponding opening of a pylorus sphincter 680 (see FIG. 14) proximate to which the device is implanted, which could potentially prevent and/or reduce the implantable liner 10 from displacement in the intestine.

In various embodiments, the proximal cuff 20 and/or distal cuff 25 may be designed with a variety of features that facilitate fixation of the implantable liner 10 to the small intestine or any digestive tract portion and desirably allow for the distal passage of appropriate digestive constituents without affecting one or more of the small intestine biomechanics, physiological response, chemical response and/or harming of the interfacing tissue. Such features may include variations in cuff length 50, cutouts 70, beveled edges 50 and/or tapered necks 80, including those as shown in FIG. 1B.

In another embodiment, the proximal cuff 20 and/or distal cuff 25 may be designed with the same or different cuff lengths 50. The cuff lengths 50 may take into consideration whether cutouts 70 are needed and/or desired, the total length 40 of the implantable liner 10 and/or relevant measurements of the patient anatomy, and/or the length needed to secure to the intestinal wall.

In another embodiment, the proximal cuff 20 and/or distal cuff 25 may include a plurality of cutouts 70. The plurality of cutouts 70 can be used to facilitate radial displacement of the device and/or tissue interfacing and/or tissue securement and/or implant adjustability relative to the native intestinal tracts. For example, the plurality of cutouts 70 may be desirably used to accommodate the insertion of a suture and/or needle to secure the proximal cuff 20 and/or distal cuff 25 to the intestinal wall. Alternatively, the plurality of cutouts 70 may be designed to allow tissue ingrowth through the cutouts 70, which can be used in addition to and/or in place of suture securement of the proximal cuff 20 and/or distal cuff 25 to the intestinal wall. The cutouts 70 may be designed in various configurations, shapes and/or sizes, as should be apparent to those of skill in the industry.

In another embodiment, the proximal cuff 20 and/or distal cuff 25 may have more, fewer or no cutouts 70 (not shown). The proximal cuff 20 and distal cuff 25 may include one or more uniform surfaces, where a surgeon or veterinarian might choose to pierce the material and secure the proximal cuff 20 and distal cuff 25 to the intestinal wall using sutures or other attachment devices as known in the art and/or described herein.

In another embodiment, the proximal cuff 20 and/or distal cuff 25 may include beveled edges 50. The beveled edges 50 may desirably facilitate atraumatic movement and/or deployment within the intestinal wall and/or easy insertion into and/or through the throat, esophagus, stomach and/or intestine. Furthermore, the beveled edges 50 may also facilitate easy passage of digested food through the cuff inner diameter 100, and potentially prevent digested food build-up.

In another embodiment, the proximal cuff 20 and distal cuff 25 may include tapered necks 80. The tapered necks 80 may desirably allow digested food to pass from the cuff inner diameter 100 to the conduit inner diameter 110 easily. The tapered necks 80 may be designed with a variety of tapered lengths and angles, including those known in the industry and/or described herein, and/or may include one or more tapered neck 80 sections to facilitate easier food digestion travel through the conduit 30.

In another embodiment, the implantable liner 10 of the implantable device, which could include one or more of the proximal cuff 20, the distal cuff 25 and/or a conduit 30, may be designed from a variety of materials. The proximal cuff 20, the distal cuff 25 and/or the conduit 30 may be manufactured using elastic, viscoelastic, and/or flexible materials to create a flexible and/or compressible/expandable device that can accommodate and/or allow for the peristaltic motion of the intestinal tract. The flexibility may include a modulus that is commensurate with the general tissue or body modulus of the digestive organ to allow for appropriate biomechanics of the peristaltic motion of the digestive organ. Such flexible materials may include silicone, polyethylene terephthalate reinforced silicones, fluoropolymers, expanded polytetrafluoroethylene (ePTFE), polycarbonate urethanes with or without functional end groups, aromatic or aliphatic polyurethanes, or polymeric fabrics such as meshes woven or knit, polyethylene terephthalate (PET) reinforced-silicone (PDMS) and/or any combination thereof. In addition, the flexible materials may be porous and/or semi-porous, if desired. The pore structure and/or physical dimensions may be designed to facilitate a desired exact amount of tissue interaction and/or barrier application, and these characteristics can be custom manipulated to accommodate a desired physiological reaction. The porosity may be designed commensurate with the size and/or diameter of one or more chemicals that is desired to allow to pass through the wall, and desirably blocking other chemicals that may be larger than and/or otherwise not fit through the pores. Specifically, the effects of such a porous barrier may directly elicit a physiological response of the blood glucose and/or insulin chemistry, or may induce an indirect effect via interference with neurohormonal pathways.

The proximal cuff 20, the distal cuff 25 and/or the conduit 30 may be manufactured with non-flexible materials, which may include component designs that may mechanically block the digestive tissue from contacting a bolus of digested food from the intestine and/or trigger a variety of biochemical responses. Such non-flexible materials may include metals, alloys, thermoset plastics, and/or any combinations thereof. Furthermore, in other embodiments, the proximal cuff 20, the distal cuff 25 and/or the conduit 30 may be manufactured using a combination of flexible and/or non-flexible materials.

In other embodiments, the proximal cuff 20, the distal cuff 25 and/or the conduit 30 flexible or non-flexible materials may be optionally chemically treated or impregnated with other constituents. Such constituents may include tissue in-growth promoting coatings, chemical coatings that may block biochemical responses, radiopathic coatings, anti-coagulant coatings, drug-eluting coatings, reduction of the coefficient of friction coatings, and/or any combination thereof.

In other embodiments, the wall thickness on the proximal cuff 20, the distal cuff 25 and/or the conduit 30 of the implant may be overall thin, locally thin, appropriately porous, and/or allow dissolving of one or more components to facilitate discharge of therapeutic chemistry and/or exchange of nutrients to the digestive system by degree and/or amount of passage, thereby eliciting degrees of effect on the biochemistry similar to those described herein.

In various additional embodiments, the proximal cuff 20, the distal cuff 25 and/or the conduit 30 may be designed to include at least one layer of flexible and/or at least one layer of non-flexible material. For example, the proximal cuff 20, the distal cuff 25 and/or the conduit 30 could comprise multiple (one or more) material layers. Where the first layer may be a flexible and/or nonporous material (i.e., calendared silicone) that provides a biomechanical neurohormonal block (such as previously described herein); the second layer may consist of a thin, flexible and tightly porous material (i.e., ePTFE); and the third layer may provide a flexible and/or non-flexible material that is impregnated with a chemical coating and/or drug-eluting coating that may be time-released for the therapeutic treatment of diabetes or obesity (e.g., drugs, gene therapies, or nutraceuticals). The one or more material layers may be coatings adhered to the wall of the proximal cuff 20, the distal cuff 25 and/or the conduit 30 and/or the one or more material layers may be independent material layers.

In other embodiments, the total length 40 of an implantable liner 10 may be designed with a length that specially affects a corresponding length of the small intestine (or any other relevant portion of the intestinal tract) that triggers one or more types of neurohormonal feedback (i.e., a biochemical response) to desirably normalize and/or otherwise elicit healthy blood glucose chemistry.

In another embodiment, the total length 50 of an implantable liner 10 can be sufficiently long to accommodate positioning within the distal stomach or antrum, with the liner extending through the duodenal bulb, and through the duodenum to the ligament of Treitz. The length of the liner and/or other components of the implantable device can vary among species and/or within species, and the total length 50 may accommodate any of the digestive and/or intestinal organs, such as the small or large intestine. In all embodiments, the length and diameter may not necessarily be limited exclusively to native organ dimensions, but may comprise a variety of lengths, sizes and/or shapes to accommodate implant stability, facilitate nutrient passage, may enable use of an appropriate delivery mechanism and can accommodate existing body biomechanics. In one exemplary embodiment, the total length 50 of an embodiment of the implant may be designed from 1 to 40 cm.

FIGS. 2A-2D depict various exemplary views of one alternate embodiment of an implantable liner 120 incorporating slotted cuffs. The implantable liner 120 may include a slotted proximal cuff 130, a slotted distal cuff 135 and a conduit 140, such as shown in FIGS. 2A and 2B. When the slotted proximal cuff 130 is magnified 150 (as shown in FIG. 2C), the magnification can highlight the slotted features, which may assist with tissue securement to the intestinal wall. In one embodiment, the slotted proximal cuff 130 and/or slotted distal cuff 135 may be designed with at least one slot 180 and beveled edge 170. The slots 180 may have a length to accommodate expansion of the tips of the cuffs, adjustability to the intestine inner diameter, folded, splayed, collapsed and/or everted positioning for attachment to the intestinal wall. The surgeon or veterinarian may desirably adjust the tips of the cuffs by mechanical expansion (i.e., stretching, balloon expansion, etc.). The slots 180 may also allow radial displacement of the cuff edges for the purpose of tissue interfacing (i.e., expansion and/or contraction during peristalsis).

In another embodiment, the slots 180 on the slotted proximal cuff 130 and/or the slotted distal cuff 135 may be positioned in different locations. For example, FIG. 2D illustrates the slots 180 in symmetric positions, such as in a 0, 90, 180, 270 degree positions. However, the slots may be asymmetrically adjacent to each other (not shown).

FIGS. 3A-3B depict various exemplary views of an alternate embodiment of the implantable liner 120 with different slotted proximal cuff 190 and/or slotted distal cuff 130 arrangements. For example, the wall thickness, cuff length, the outer diameter, the cutouts, the slot configuration, the slot length, and/or the removal of slots may differ between the slotted proximal cuff 190 and the slotted distal cuff 130.

In various alternative embodiments, the differentiation between the proximal and distal cuff ends can include a wide variety of design features, and are not necessarily limited to the slotted cuff design. FIGS. 4A-4B depict various exemplary views of an alternate embodiment of the implantable liner 200 with differentiated proximal and distal cuff retaining mechanism designs. The proximal and distal cuff retaining mechanism designs may be utilized for a variety of reasons, including being designed using materials that promote tissue ingrowth and/or provide radiopacity during surgery, which may allow for increased surface area or differing attachment methodologies for intestinal (or other organ) wall attachment, which may serve as one or more retaining mechanisms for use with the delivery system, which may serve to resist radial forces on the implantable liner occurring due to the peristaltic traction from the intestinal wall (or other physiological factors) on the liner, which may be used to retain sutures, materials, anchors, and/or wires to facilitate deployment and/or securement to the intestinal wall, or for a variety of other design reasons. Furthermore, if desired the proximal and/or distal cuff retaining mechanism designs may be removable, modular, and/or fixed to the implantable liner.

FIG. 4A depicts an isometric view of one embodiment an implantable liner 200 that includes a curvature 235, a pledget cuff 220, an anchor cuff 210 design, and/or a conduit 215. The curvature 235, as best shown in FIG. 4B, may be any configuration that could facilitate the implantable liner 200 to accommodate any intestinal tortuosity, including replication of the existing anatomy as well as designs that alter the existing anatomy in a desired manner. For example, the surgeon may desirably prefer the implantable liner 200 to have a curvature 235 that may accommodate the tortuosity 740 of the small intestine (see FIG. 14) where the jejunum transitions to the duodenum. The curvature may include any configuration, radius, curvature, diameter that will easily be deployed and accommodate the shape, size, length and/or configuration of the tortuosity of the intestinal tract. Also, the proximal liner length 240 and the distal liner length 230 may be designed with equivalent lengths or differentiated lengths, where the curvature 235 may be centered or off-centered.

FIG. 4C depicts a magnified isometric view of one embodiment of a pledget cuff 200 retaining mechanism. The implantable liner 200 pledget cuff 220 may be designed with a upper portion stop 310, a pledget ring 300 that sits over the pledget cuff outer diameter 292, and a lower portion stop 290. The upper portion stop 310 may be designed with a tapered tip to allow easy insertion of the pledget ring 300. In addition, the upper portion stop 310 may include an inner diameter 294 that may be equal to and/or different than the inner diameter of the implantable liner 200 conduit 215. The outer diameter of the upper portion stop 310 and/or the outer diameter of the lower portion stop 290 may be designed to accommodate any diameter that allows approximation to the intestinal wall, which may include dimensions larger than the inner diameter 380 (see FIG. 6B) of the pledget ring 300, to desirably prevent movement of the pledget ring 300 during delivery and/or deployment. The pledget ring outer diameter 370 may be any diameter that can accommodate the intestinal wall inner diameter (see FIG. 6B) and may include beveled 390, tapered (not shown), and/or chamfered (not shown) edges to facilitate easy loading onto the implantable liner 200 and/or to prevent tissue damage by use of atraumatic designs (see FIG. 6A). The pledget may be geometrically square, round, or any polygon for use in discreet locations around the circumference of the intestine as opposed to a continuous pledget body.

FIG. 4D depicts a magnified isometric view of one embodiment of an implantable liner 200 anchor cuff 210 retaining mechanism. The anchor cuff 210 may include an upper portion stop 250, a grommet 270, and/or one or more anchor members 260 with one or more anchor protrusions 280. In this embodiment, prior to deployment of the implantable liner 200 in the intestinal tract, the grommet 270 can be loaded onto the one or more anchor members 260 and be seated adjacent to the one or more anchor protrusions 280. Once the implantable liner 200 is inserted into the intestinal tract, the anchor cuff 210 may be positioned anywhere along the wall of the targeted length of the intestinal tract and/or stomach, including within the stomach above the pyloric sphincter 680 (see FIG. 14), and/or proximate to any reduced diameter opening in the intestinal tract (not shown). To deploy the anchor, the grommet 270 may be slid, pushed and/or pulled towards the upper portion stop 250, thereby deploying the one or more anchor members 260 radially outward, which may allow one or more of the anchor protrusions 280 to grasp and/or retain onto the intestinal wall or other surrounding structure, and/or prevent axial movement in various manners. The anchor protrusions 280 may be designed with a variety of shapes, including those well known in the industry, to help with tissue approximation, tissue grasping, and/or preventing axial movement (i.e., barbs, “T” shapes, roughened surfaces, coated surfaces, etc. . . . ). Furthermore, the grommet 270 may include other features, such as counterbores 350 (see FIG. 5B), where the anchor protrusions 280 may be loaded through the one or more openings 320 (see FIG. 5A) and seated onto the counterbore 350. In addition, in some embodiments, the anchor cuff 210 may cylindrically interface with tissue, and be fixedly attached yet reversible in at least one location.

GI Delivery System

FIG. 7 depicts an isometric view of one embodiment of a GI delivery system 400. The GI delivery system 400 may be designed as modular and/or one-piece fixed design. The GI delivery system may come equipped with a handle 410, at least one attachment rods 420, and/or at least one nose cone 430.

FIG. 8A depicts a side view of the GI delivery system handle 410. The handle 410 may be designed with various shapes and sizes. The handle length 440 and the handle outer diameter 460 (see FIG. 8B) can be designed to accommodate standard surgeon and/or veterinarian sized hands, and/or any length and/or outer diameter known in the art that would allow easy grasping and manipulation. The handle tip 450 may include beveled, chamfered, and/or tapered tips for atraumatic insertion into the intestinal tract. Furthermore, the handle 410 may also include grasping features 470 that may run axially along the length 440 of the handle 410. The grasping features 470 may be any shape and sizes, such as channels, roughened surfaces, detents (concave or convex), and/or any combination thereof.

FIG. 8C depicts a bottom plan view of the GI delivery system handle 410. The bottom view highlights the opening 480 where the at least one attachment rod 420 may be inserted. The opening 480 may be designed to accommodate the outer diameter of the at least one attachment rod 420 and may be secured to the handle 410 by a variety of mechanisms known in the art (i.e., threads, press-fit, over-molding, tabs, detents, etc. . . . ).

FIGS. 9A-9B depicts various exemplary views of one embodiment of GI delivery system attachment rods 420. The GI delivery system 400 may include at least one attachment rod 420. The attachment rod length 490 may be designed to accommodate the implantable liner, the at least one nose cone 430, and/or a combination thereof. The attachment rod 420 may have dual sided securing mechanisms 500 that may be used to removably connect and/or permanently fix to the handle 410 and the at least one nose cone 430. Such securing mechanisms may be known in the art (i.e., threads, press-fit, over-molding, tabs, detents, etc.). Furthermore, the attachment rod outer diameter 495 may be designed to accommodate the conduit inner diameter of the implantable liner. The attachment rods 420 may be designed from flexible and/or non-flexible materials as desired from the surgeon or veterinarian. Consideration to the type of materials may be based on tortuosity of the intestinal tract, surgical procedure/technique (i.e., open vs. endoscopic), sterilization, and many other factors known in the art.

FIGS. 10A-10C, 11A-11C, and 12A-12C depicts various exemplary views of a large nose cone 430, a medium nose cone 580 and a small nose cone 600. The various embodiment of nose cones described herein include various novel holding features that desirably allow tactile interpretation and location of the correct position to secure the implant, including the use of such devices through at least one body orifice and/or opening (or artificially created openings) and/or may be used to temporarily hold the implantable liner cuffs in one or more desired positions prior to delivery and securement of the implantable device within the intestinal tract. The holding feature may include a variety of securing features, retaining features, connecting features, and/or fixing features, which may all facilitate the positioning and/or securing of the implantable device to the GI delivery system 400 and/or to the surrounding tissue and/or biological constituent(s).

FIGS. 10B, 11B and 12B best show how the large nose cones 430, the medium nose cone 580 and the small nose cones 600 include various holding features 520, 590 and 610. These holding features may be constructed in a variety of shapes and/or sizes, such as hemispheres, squares, triangles, polygons, and/or any combinations thereof. Furthermore, other embodiments of the holding features may include openings 597 and/or channels that could be used to insert fibers, sutures, and/or any connection mechanism(s) through the opening and/or channels. The holding feature outer diameter 550, 595, 615 may be formed in a variety of dimensions that may facilitate the securement of the varying sized inner diameters of the implantable liner conduits and/or implantable liner cuffs. Securement of the implantable liner conduit and/or the implantable liner cuff may be accomplished by friction, use of fibers, sutures, elastic materials, or viscoelastic retainer or connector mechanisms that may use inert and/or dissolvable materials.

In other embodiments, the large nose cone 430, medium nose cone 580 and/or small nose cone 600 may contain troughs or scooped channels 530, 592, 612 that may be particularly useful during attachment and/or anchoring of the device to the surrounding anatomy, allowing for needle guidance, needle penetration feedback, and/or needle redirection. The troughs and/or scooped channels 530, 592, 612 may be designed to include a variety of shapes and/or configurations that accommodate a variety of differing types of needles, tools and/or other instruments used by a physician and/or veterinarian to deploy and secure the implantable liner within the intestinal tract.

In some embodiments, the GI delivery system 400 is fabricated as a single fixed unit or multiple (modular) components assembled in a single step such as injection molding or by means of rapid prototyping technologies not limited to Stereolithography (SLA), Selective Laser Sintering (SLS), Fused Deposition Modeling (FDM), Direct Metal Laser Sintering (DMLS) and Metal Injection Molding (MIM). Furthermore, each of the components within the GI delivery system 400 may be manufactured from a variety of materials, such as metals, polymers, alloys, flexible, non-flexible, porous materials and/or any combination thereof.

FIGS. 13A-13B depict various exemplary views of the GI delivery system of FIG. 7 with an associated implantable liner 620 deployed thereupon. In various other embodiments, the GI delivery system 400 and the various embodiments of the implantable liner could be manufactured by implementing lean manufacturing and demand flow principles. Such lean manufacturing and demand flow principles may include the implementation of ISO and FDA practices outlined and described in ISO 13485 and FDA cGMP. Furthermore, the GI delivery system 410 and the various embodiments of the implantable liner can be assembled (but not necessarily limited to such assembly) in a controlled environment such as white room, laboratory, controlled environment room, cleanroom, clinical environment, or operating room. In a preferred embodiment, the implant device, the delivery system, and associated packaging are delivered to the end user as a sterile or unsterile kit or single item packaging in the same sterile or unsterile manner. Sterilization could be achieved by appropriate means based upon material and design constraints, including sterilization methods such as ETO gas, heat, chemical or radiation sterilization.

In some embodiments, the GI delivery system 400 can be available in different sizes, as desired by the surgeon and/or veterinarian. The different sizes of the GI delivery system 400 could be derived and/or determined using data of standard sized intestinal tract dimensions, and/or derived from specific measurements from the specific species. The different sizes of the GI delivery system 400 can be available in a kit, where the entire one-piece fixed GI delivery system 400 may be provided in different sizes, i.e., small, medium, and/or large. Alternatively, the different sizes of the GI delivery system 400 may allow for substitution of the different modular components, such as the attachment rods 420, the nose cones (430, 580, and 600), and the various embodiments of the implantable liners. The different modular components may be assembled, substituted and/or replaced in-vivo during surgery.

GI Delivery System Surgical Method

In some embodiments, the GI delivery system with the implantable liner 620 (see FIGS. 13A and 13B) may be implanted in the targeted intestinal tract through various surgical procedures. Such surgical procedures may include invasive open surgical procedure, laparoscopic, endoscopically and/or minimally invasive procedures.

The surgeon or veterinarian may receive a kit for the procedure that may include a GI delivery system 400 (see FIG. 7), various sizes and/or configurations of implantable liners, and various sizes of nose cones (i.e., small, medium, and/or large). The surgeon or veterinarian may choose to conduct a laparotomy (not shown) followed by either an enterotomy incision 640 or gastrotomy incision 650 (see FIG. 14). If the surgeon desirably proceeds with an enterotomy, the enterotomy incision 640 could be placed distal to the pylorus 690. Alternatively, if the surgeon desirably proceeds with a gastrotomy, the gastrotomy incision 650 could be placed on the proximal side of the pylorus 690.

Once the targeted incision is made, the surgeon or veterinarian may access the intestinal tract to determine the proper size device and/or delivery system desired to approximate the inner diameter of the intestinal tract. The surgeon or veterinarian may decide to insert one or more of the various sizes of nose cones (i.e., small, medium, and/or large) into the inner diameter of the intestinal tract, i.e., the duodenum 720, to determine the proper sized nose cone and implantable liner.

The surgeon and/or veterinarian may assemble the proper sized nose cones onto the attachment rods and handle, then may subsequently load the proper sized implantable liner. The implantable liner may be loaded onto the nose cones in a concentric manner, which could ensure that the implantable liner cuffs covered the holding features on the nose cones to provide proximal and/or distal radial and/or axial tension, by radially stretching the implantable liner cuffs. The implantable liner might not require radial alignment to the nose cones. However, the implantable liner may require radial registration of the distal and proximal cuffs to ensure no mid-length twisting. The surgeon and/or veterinarian may confirm that the implantable liner cuffs may cover the holding features, thereby allowing the most proximal and distal cone tapers of the nose cones to be exposed. The holding features on the nose cones could be designed to radially tension the implantable liner cuffs, thereby desirably holding the device onto the delivery system during passage into the enterotomy and luminal transit to the final implant location in vivo.

The surgeon and/or veterinarian may begin to insert the GI delivery system with the implantable liner 620 into the intestinal tract. The GI delivery system with the implantable liner 620 can be distally positioned beyond the bile duct 710 and the pancreatic duct 730 terminating proximal to the jejunum 740 (see FIGS. 14 and 15). Alternatively, the GI delivery system with the implantable liner 620 may have at least one implantable liner cuff positioned above the pyloric sphincter 680, where the other implantable liner cuff could be positioned to terminate proximal to the jejunum 740 and/or distal to the jejunum 740. Once a desired final position for the device has been reached, the surgeon and/or veterinarian may use tactile feedback to palpitate the holding features on the nose cones from the exterior of the intestine. Alternatively, if 2D or 3D imaging is available, the surgeon and/or veterinarian may choose to image to locate and/or position the implant. Upon determining the location of at least one of the holding features on the nose cones, the surgeon and/or the veterinarian may secure the proximal and/or distal implantable liner cuff using one or more suture stitches through the intestinal wall (or other relevant anatomy) while using the trough(s) on the nose cones as suture needle guides.

The surgeon and/or the veterinarian may continue to palpitate the intestinal wall to locate the proximal and distal nose cones to add additional sutures for securement of the implantable liner cuffs to the intestinal wall. The palpitation and suturing process may be generally repeated in 4 or more radial locations, include locations paired or tripled radially and linearly within the trough area of the nose cones.

Once the implantable liner cuffs are proximally and distally secured by one or more sutures into the intestinal wall, the GI delivery system can be retracted or withdrawn by gently rotating and/or pulling simultaneously. An alternate option could be to only distally secure the implant with silicone bands that might gain purchase through the implantable liner cuff by the troughs formed in the nose cones. These bands could fall off after suture securement. Another alternative could be to secure the implant with restorable bands that may or may not inconsequently tangle with securing suture, but rather may dissolve and render the implant secure.

Although sutures may be used to secure the implantable liner cuffs to the intestinal wall, the implantable liner can include features that allow removable attachment and/or securement, or such components could be secured permanently. If the implantable liner is removably attached, the surgeon and/or veterinarian may facilitate removal and/or replacement of the implantable liner. The second implantable liner might have a longer length or shorter overall length, larger or smaller inner diameter conduit, larger or smaller outer diameter conduit, longer or shorter implantable liner cuffs, larger or smaller cuff diameters, and/or any combination thereof. This adjustability may be necessary if the first implantable liner was suboptimal or becomes suboptimal due to post implantation changes such as stomach remodeling, therapeutic changes and/or behavioral changes. Such revision may also be necessary and/or advised where movement of the implant and/or associates cuffs occurs post operatively.

In addition to the various disclosures described herein, Applicants' disclosure expressly incorporates by reference the disclosures of U.S. Pat. No. 7,025,791 entitled “Bariatric Sleeve,” and U.S. Pat. No. 7,122,058 entitled “Anti-Obesity Devices.” These references, as well as any other references cited herein, including publications, patent applications, and patents, are hereby incorporated by reference to the same extent as if each reference were individually and specifically indicated to be incorporated by reference and were set forth in its entirety herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A conduit for implantation into a gastrointestinal tract of a subject, comprising:
a substantially flexible, elongated tubular body having a longitudinally-extending central lumen, a proximal end and a distal end;
a first tubular cuff connected to the proximal end, the first tubular cuff including a first cuff opening extending through the first tubular cuff and in fluid communication with the longitudinally-extending central lumen of the tubular body, the first tubular cuff being sized and configured to fit within a first location within the gastrointestinal tract of the subject;
a second tubular cuff connected to the distal end, the second tubular cuff including a second cuff opening extending through the first tubular cuff and in fluid communication with the longitudinally-extending central lumen of the tubular body, the second tubular cuff being sized and configured to fit within a second location within the gastrointestinal tract of the subject;
a wall of the substantially flexible, elongated tubular body comprising an inner layer of flexible, substantially nonporous material and an outer layer of flexible, substantially porous material; and
the outer layer of substantially porous material impregnated with a therapeutic constituent.

2. The conduit of claim 1, wherein at least one of the first or second tubular cuffs comprises a plurality of apertures configured to receive sutures.

3. The conduit of claim 1, wherein the elongated tubular body, the first tubular cuff and the second tubular cuff each comprise a material having a modulus of elasticity that approximates a native modulus of the gastrointestinal tract during peristaltic motion.

4. The conduit of claim 1, wherein the inner layer and outer layer comprise two independent material layers adjacent to each other.

5. The conduit of claim 1, wherein the wall of the substantially flexible, elongated tubular body further comprises a central layer of flexible substantially nonporous material.

6. The inner layer of claim 4, wherein the inner layer comprises of calendared silicone.

7. The outer layer of claim 4, wherein the outer layer comprises of PET woven mesh fabric.

8. A gastrointestinal implant and delivery system comprising:
an implantable liner, the implantable liner having a substantially flexible proximal cuff, a substantially flexible distal cuff, and a substantially flexible conduit with a longitudinally extending length between the proximal cuff and distal cuff; an opening extending through the proximal cuff to the distal cuff;
a delivery system; the delivery system having a handle, an attachment rod, a first nose cone and a second nose cone;
the first nose cone having a longitudinal extending body, the longitudinal extending body having an outer surface and at least one guide channel recessed from the outer surface;
the second nose cone having a longitudinal extending body, the longitudinal extending body having an outer surface and at least one guide channel recessed from the outer surface;
the attachment rod having a longitudinally extending length with a first end and a second end, the first nose cone connected to the attachment rod a first location proximate to the first end of the attachment rod; and
the second nose cone connected to the attachment rod at a second location, a longitudinal spacing between the first nose cone and a second nose cone approximating the longitudinally extending length of the substantially flexible conduit.

9. The gastrointestinal implant and delivery system of claim 8, wherein the first nose cone is removably connected to the attachment rod.

10. The gastrointestinal implant and delivery system of claim 8, wherein the second nose cone is removably connected to the attachment rod.

11. The gastrointestinal implant and delivery system of claim 8, wherein the attachment is removably connected to the first nose cone and the second nose cone.

12. The gastrointestinal implant and delivery system of claim 8, wherein the second location is the second end of the attachment rod.

13. The gastrointestinal implant and delivery system of claim 8, wherein the at least one guide channel extends in a longitudinal direction along the longitudinal extending body of the first nose cone.

14. The gastrointestinal implant and delivery system of claim 8, wherein the at least one guide channel extends in a longitudinal direction along the longitudinal extending body of the second nose cone.

15. The gastrointestinal implant and delivery system of claim 8, wherein the longitudinal extending body of the first nose cone includes at least one holding feature protruding from the outer surface of the first nose cone, the at least one holding feature being sized and configured to accommodate an inner diameter of the opening extending through the proximal cuff.

16. The gastrointestinal implant and delivery system of claim 8, wherein the longitudinal extending body of the second nose cone includes at least one holding feature protruding from the outer surface of the second nose cone, the at least one holding feature being sized and configured to accommodate an inner diameter of the opening extending through the proximal cuff.

17. The gastrointestinal implant and delivery system of claim 15, wherein the longitudinal extending body of the first nose cone includes a plurality of holding features protruding from the outer surface of the first nose cone, the plurality of holding features being sized and configured to accommodate an inner diameter of the opening extending through the proximal cuff.

18. The gastrointestinal implant and delivery system of claim 16, wherein the longitudinal extending body of the second nose cone includes a plurality of holding features protruding from the outer surface of the second nose cone, the plurality of holding features being sized and configured to accommodate an inner diameter of the opening extending through the proximal cuff.

19. A method of deploying an implantable liner into a gastrointestinal tract comprising the steps of:
measuring an inner diameter of the gastrointestinal tract at a first location and a second location;
selecting an implantable liner having a longitudinal length that is sized and configured to extend from the first location to the second location, the implantable liner having a first cuff and a second cuff, the first cuff being sized and configured to fit within the inner diameter of the gastrointestinal tract at the first location, the second cuff being sized and configured to fit within the inner diameter of the gastrointestinal tract at the second location;
selecting a delivery system for carrying the implantable liner, the delivery system having a first nose cone and a second nose cone, the first and second nose cones each including at least one guide channel;
inserting the implantable liner and the delivery system into the inner diameter of the gastrointestinal tract;
positioning the implantable liner and the delivery system within the gastrointestinal tract wherein the first cuff is positioned proximate to the first location within the gastrointestinal tract and the second cuff is positioned proximate to the second location within the gastrointestinal tract;
securing the first cuff of the implantable liner to a wall of the gastrointestinal tract at the first location using the guide channel on the first nose cone;
securing the second cuff of the implantable liner to the wall of the gastrointestinal tract at the second location using the guide channel on the second nose cone; and
withdrawing the delivery system to leave the implantable liner within the gastrointestinal tract.

20. The method of claim 19, wherein the second location is proximal to the jejunum.
